(19) United States
(12) Patent Application Publication
(10) Pub. No.: US 2007/0123963 A1
(43) Pub. Date: May 31, 2007

(54) Method for producing flexible, stretchable, and implantable high-density microelectrode arrays

(76) Inventor: Peter Krulevitch, Pleasanton, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/288,759

(22) Filed: Nov. 29, 2005

(51) Int. Cl. A61N 1/00

(52) U.S. Cl. 607/115

(57) ABSTRACT

A high-density microelectrode array that is flexible and stretchable and can also be implanted within living tissue is provided. The microelectrode array includes at least first and second implantable and biocompatible polymeric layers in which a plurality of patterned conductive features, including metallic contact pads, metallic traces and metallic electrodes are sandwiched therebetween. Each metallic trace is located between a metallic contact pad and a metallic electrode and has substantially rounded corners and a zigzag pattern. The latter features are provided using stent technology. The present invention also provides a method of fabricating such a flexible, stretchable, and implantable microelectrode arrays which combined micromaching technology and stent technology as well as an implantable medical device that includes the inventive microelectrode array.

### FIELD OF THE INVENTION

The present invention relates to electrodes and more particularly to a high-density microelectrode array that is flexible and stretchable and can also be implanted within living tissue. The present invention also provides a method of fabricating such a flexible, stretchable, and implantable microelectrode array as well as an implantable medical device that includes the inventive microelectrode array.

### BACKGROUND OF THE INVENTION

Microelectrode arrays are currently being developed for a broad range of applications including, for example, for use in various implantable medical devices. Implantable medical devices are defined herein as a physical article used in medical treatment that can be introduced into living tissue. Some examples of medical devices, which can contain microelectrode arrays, include, for example, cochlear implants, visual prostheses, neurostimulators, muscular stimulators, and deep brain stimulators.

A typical microelectrode array consists of multiple micron to mm scale electrodes with conducting traces and contact pads for interfacing to driving electronics. The conductive traces (or conductive wires or lines) are used to connect the electrodes of the array to the contact pads, which, in turn, are used to interface with the driving electronics of the medical device.

Most of today's medical devices that are approved by the U.S. Food and Drug Administration include microelectrode arrays that comprise bulk platinum (Pt) traces and electrodes embedded within a polymer body (or matrix), which are manually assembled using conventional (i.e., non-microfabrication) techniques well known in the art. The polymer body of such arrays is typically comprised of silicone or polyurethane.

Recent experimental medical devices take advantage of microfabrication techniques such as photolithographic patterning of metal films and electroplating of metal films to produce microelectrode arrays with smaller feature sizes and a greater number of electrodes than traditional microelectrode arrays. These prior art microelectrode arrays typically use silicon or a polyamide substrate, thin film Pt traces and thicker electrode plated Pt electrodes. Recently, microelectrode arrays with silicone substrates and stretchable thin film gold traces have been developed. Such arrays are disclosed, for example, in U.S. Pat. No. 6,878,643 as well as U.S. Patent Application Publication Nos. 2003/0097166 A1, 2003/0097165 A1, 2004/0243204 A1, 2004/0238819 A1, and 2005/0030698 A1.

Problems exist with all the approaches mentioned above. For example, silicon and polyamide, while compatible with micromachining processes, are not sufficiently compliant to meet application needs, and electroplated platinum is susceptible to cracking and delamination due to large residual stresses. While the techniques disclosed in the aforementioned U.S. patents and U.S. patent application publications are promising, thin gold traces are not acceptable, and producing high quality thick Pt electrodes on silicone using standard deposition techniques is extremely challenging. Also, many of the prior art microelectrode array designs are not flexible and stretchable enough to be used with current implantable medical devices.

In view of the drawbacks mentioned above with fabrication of prior art microelectrode arrays, there is still a need for providing an alternative method of fabricating microelectrode arrays that are flexible, stretchable and can be implanted safely within living tissue.

### SUMMARY OF THE INVENTION

The present invention provides an alternative approach for fabricating a microelectrode array that combines micromachining techniques with methods used for producing metal stents. The method of the present invention utilizes materials that are compatible with micromachining processes, and the materials are sufficiently compliant to meet current needs for use as a component of an implantable medical device.

In accordance with the present invention, a first implantable and biocompatible polymeric layer is formed on a surface of a handle substrate. The first polymeric layer is then cured providing a cured first polymeric layer on the handle substrate. A carrier substrate including a plurality of patterned conductive features comprising metallic contact pads, metallic traces and metallic electrodes is formed. In accordance with the present invention and within the array, a single metallic electrode is contacted to a single metallic contact pad by a single metallic trace. In some embodiments of the present invention, it is possible that there could be more than one electrode associated with a single contact pad.

Each of the metallic traces of the patterned conductive features are patterned to have a zigzag (or serpentine) configuration with substantially rounded corners similar to designs used for expandable stents to allow for stretching of the microelectrode array. The metallic traces having this zigzag pattern and substantially rounded corners provide an electrical contact between neighboring metallic electrodes and metallic contact pads. The patterned conductive features are then transferred to the first polymeric layer using bonding techniques and at least the carrier substrate is removed at this point of the inventive process to expose the surface of the first polymeric layer including the patterned conductive features. In some embodiments of the present invention, the conductive traces are transferred to the first polymeric layer with bonding, and the traces are held in place when the second polymeric layer is applied.

A second polymeric layer, that is also implantable and biocompatible, is then formed on the bonded structure such that the patterned conductive features are encapsulated (i.e., surrounded or encased) within the polymeric layers. It is noted that the polymeric layers used in the present invention are insulating materials that are generally hydrophobic. The second polymeric layer may be pre-patterned prior to forming on the bonded structure or the second polymeric layer may be patterned after application to the bonded structure. The patterns formed into the second polymeric layer are typically vias (i.e., openings) that extend down to the first patterned conductive features exposing the metallic contact pads and metallic electrodes. The patterns also define the shape of the microelectrode array. The vias can be filled with a conductive material and contacts can be made with other elements or components of an implantable medical device.

The above steps can be repeated numerous times to create multiple layers of metal with alternating polymeric layers to produce multi-layer three-dimensional stacks with increased number of electrodes. After all the metal and polymeric layers are formed, the devices are sectioned and removed from the carrier substrate utilizing conventional techniques well known in the art.

In addition to the method described above, the present invention also provides a microelectrode array that is useful in implantable medical devices. The inventive microelectrode array includes at least first and second implantable and biocompatible polymeric layers in which a plurality of patterned conductive features including metallic contact pads, metallic traces and metallic electrodes is sandwiched therebetween, wherein each metallic trace has a zigzag pattern and substantially rounded corners.

In addition to the array, the present invention also provides an implantable medical device which comprises at least the microelectrode array of the present invention.

### BRIEF DESCRIPTION OF THE DRAWINGS

FIGS. 1A-1E are pictorial representations illustrating the basic processing steps of the present invention; FIGS. 1A-1B and 1D-1E are cross sectional views, while FIG. 1C is a top down view.

FIG. 2 is a pictorial representation (pseudo-3D) showing a basic microelectrode array structure of the present invention.

### DETAILED DESCRIPTION OF THE INVENTION

The present invention, which provides a method of fabricating flexible, stretchable and implantable microelectrode arrays as well as the microelectrode arrays themselves, will now be described in greater detail by referring to the following discussion and drawings that accompany the present application. The drawings, which are included with the present application, are provided for illustrative purposes and, as such, they are not drawn to scale. For example, in FIG. 2 the metal layer would be much thicker than that which is shown and the polymeric layers would be much thinner than that which is shown.

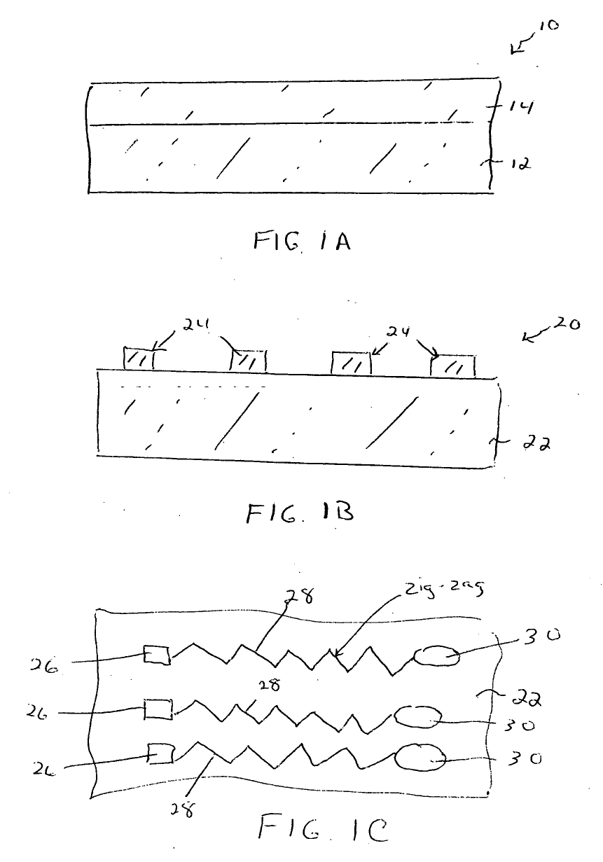

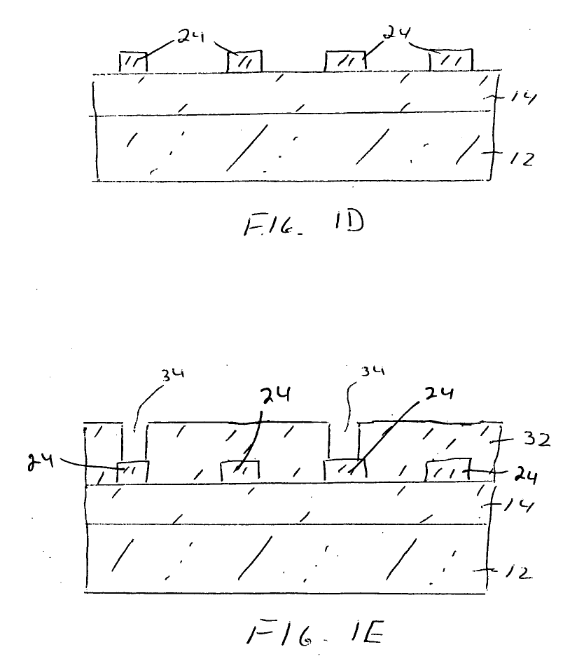

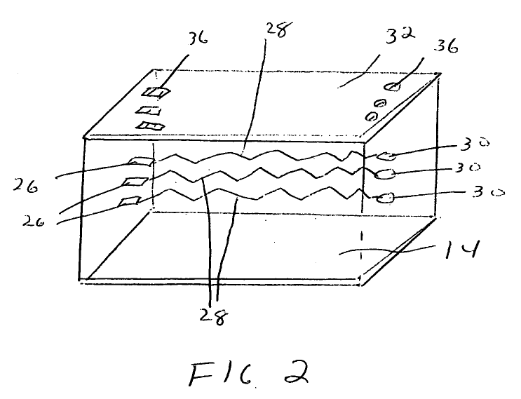

The method of the present invention begins with providing the two structures shown in FIG. 1A or 1B. The two structures can be prepared in any order and, as such, the present invention is not limited to the order specified in the drawings. FIG. 1A shows a first structure 10 that includes a handle substrate 12 and a cured first implantable and biocompatible polymeric layer 14 located thereon. The handle substrate 12 may comprise a Si wafer, glass, plastic, ceramic or multilayers thereof. Typically, a Si wafer is used as the handle substrate 12 since they are flat, stable, routinely used in microfabrication applications and they are readily available. In some embodiments of the present invention, a non-stick layer (not shown) can be applied to the handle substrate 12 prior to forming the first polymeric layer 14 thereon.

The first polymeric layer 14 is applied to an upper exposed surface of the handle substrate 12 utilizing a conventional deposition process including, for example, spin-on coating, spray coating, dip-coating, casting, or vapor deposition (for parylene). Typically, a spin-on coating process is used to apply the first polymeric layer 14 to the handle substrate 12.

Notwithstanding the deposition technique used, the first polymeric layer 14 has an as-deposited thickness that is typically from about 1 to about 500 microns, with a thickness from about 10 to about 50 microns being even more typical.

The first polymeric layer 14 is comprised of any implantable and biocompatible polymer. By “implantable” it is meant that the polymeric material can be inserted into a living site for medical usage. The term “biocompatible” denotes that the polymeric material is compatible with a living tissue or a living organism by not being toxic or injurious and by not causing immunological reaction. The polymeric material employed in the present invention is generally a hydrophobic material that is flexible and which can conform to many different shapes, including curved surfaces. It is noted that the term ‘polymer’ is used to denote a chemical compound with high molecular weight consisting of a number of structural units linked together by covalent bonds.

Illustrative examples of polymeric materials that can be used in the present invention as the first polymeric layer 14 include, but are not limited to: silicone polymers (i.e., organosiloxanes), polyurethanes, polyamides, parylene, fluoropolymers such as, for example, Teflon, polyolefins such as, for example, polyethylene and polypropylene, collagen, chitin, alginate polyvinyl pyrrolidone, polyethylene glycol, polyethylene oxide, polyvinyl alcohol, polyglycol lactic acid, polylactic acid, polycaprolactone, polyamino acid, and a hydrogel such as, for example, carboxymethyl cellulose.

In one preferred embodiment of the present invention, the first polymeric layer 14 is a silicone polymer. Silicone polymers generally are characterized as having the formula (RₙSiO_{4-n/2}) wherein R is an organic group, n is 1-3, and m is greater than or equal to 2. A silicone polymer contains a repeating silicon-oxygen backbone and has organic groups R attached to a significant proportion of the Si atoms by silicon-carbon bonds. In many of the commercially available silicones, most of the R's can be an alkyl containing from 1 to about 20 carbon atoms, fluoroalkyl, phenyl, vinyl, and some of the remaining R's can be hydrogen, chloride alkoxy, acyloxy or alkylamine.

In this preferred embodiment of the present invention, the first polymeric layer 14 includes one of poly(dimethylsiloxane), polyurethane, parylene, and the like. Of the various polymers mentioned above, poly(dimethylsiloxane) (PDMS) is highly preferred in the present invention. PDMS has low water permeability and protects electronic components from the environment. Also, PDMS is very flexible and will conform to curved surfaces. Additionally, PDMS is transparent, stretchable, resinous, rubbery and provides numerous applications for the microelectrode array of the present invention.

Curing of the as-deposited first polymeric layer 14 is performed at a temperature from about 20° to about 100° C. for a time period from about 0.5 to about 48 hours. The curing temperature and time will vary depending on the material of the first polymeric layer 14 as well as the thickness of the as-deposited layer. Typically, and for PDMS having a thickness within the above range, a curing temperature of about 66° C. for a time period from about 24-48 hours is employed. As is known to those skilled in the art, curing polymerizes the polymer.

FIG. 1B shows a second structure 20 that includes a carrier substrate 22 having a plurality of conductive features 24 located on a surface thereof. The carrier substrate 22 may comprise the same or different material as that of the handle substrate 12.

The term “conductive features” is used throughout this application to denote metallic electrodes, metallic traces, and metallic contact pads. In accordance with the present invention, the metallic traces provide electrical contact between neighboring metallic pads and metallic electrodes. FIG. 1C shows a top down view showing a plurality of metallic electrodes 30, metallic traces 28 and metallic contact pads 26 arranged in the manner indicated above. It should be noted that more than one metallic electrode 30 can be associated with a single metallic contact pad 26.

In accordance with the present invention, the plurality of conductive features 24 is formed such that each of the metallic traces 28 has a zigzag (or serpentine) pattern and substantially rounded corners. This design for the metallic traces is similar to those found in many medical stents and it also allows for stretching of the inventive microelectrode array. That is, each of the metallic traces 28 present in the inventive microelectrode array is arranged such that it has sharp turns and angles that alter the course of the metallic trace. The number of turns and angles present in each metallic trace 28 may vary depending on the total area of the final device. Each metallic trace 28 must, however, have at least one turn and angle that changes the course of the metallic trace connected a metallic contact pad to a metallic electrode. Typically, each metallic trace 28 is designed to contain from about 2 to about 200 turns and angles, depending on the length of the device. The term “substantially rounded” is used herein to denote a radius of curvature greater than approximately the width of the trace.

The term “metallic” is used in the present invention to denote a material that includes at least one metal or metal alloy that is conductive. Illustrative examples of metallic materials that can be used in forming the plurality of conductive features 24 include, but are not limited to: Pt, Ti and alloys such as an alloy of NiTi. In one embodiment of the present invention, Pt is used as the metallic material of the least one conductive feature 24. In another embodiment of the present invention, an alloy of NiTi known as Nitinol supplied by Nitinol Devices and Components can be used since this alloy is superelastic and thus can provide metallic traces 28 that are capable of exhibiting extremely large deformations. It is noted that in the present invention a single metallic foil or sheet can be used to provide the plurality of conductive features 24 which is an advantage over some of the prior art in which multiple films are used in creating such features.

The plurality of conductive features 24 can be formed utilizing laser machining in which a metallic foil or sheet is first applied to a surface of the carrier substrate 22. The foil or sheet can be patterned by laser cutting (like stents) or wet chemical etching.

The thickness of metallic foil or sheet formed at this point of the present invention may vary and can be determined by the skilled artisan. Typically, the metallic foil or sheet formed at this point of the present invention has thickness from about 5 to about 500 microns, with a thickness from about 10 to about 75 microns being more typical.

After applying the metallic foil or sheet to the surface of the carrier substrate 22, the plurality of conductive features 24 is formed by laser machining. Laser machining is generally a technique that is used in fabricating medical stents and is thus well known in medical device fabrication. Typically, laser machining is performed utilizing a laser system that is scanned over the substrate, ablating material when the laser energy contacts the substrate.

In another embodiment of the present invention, the plurality of conductive features 24 is formed utilizing photolithography and etching. The term “photolithography” is used throughout this application to denote a patterning technique in which a photoresist (either positive-tone or negative-tone) is applied to the upper exposed surface of a film needing patterning. The photoresist can be applied by utilizing any deposition technique, with spin-on coating, dip-coating, and spray coating being highly preferred. Following the application of the photoresist, the photoresist is exposed to a pattern of radiation. In the present invention, the pattern of radiation allows for the formation of the plurality of conductive features 24. After radiation exposure, the exposed resist is developed utilizing a conventional resist developer. The lithographic step thus forms a patterned photoresist having the pattern of the plurality of conductive features 24 located therein. Because of the nature of the photolithographic process the pattern formed into the resist has inherent corner rounding. This pattern is then transferred to the metallic film utilizing an etching process. The etching process may include a dry etching technique such as, for example, reactive-ion etching (RIE), ion beam etching, plasma etching or laser ablation. Alternatively, the etching can be achieved utilizing a chemical wet etching process in which a chemical etchant that selectively removes the exposed portions of the metallic film is used. After pattern transfer via etching, the patterned photoresist is removed utilizing a conventional stripping process well known to those skilled in the art.

In some embodiments of the present invention, the conductive features are transferred to the first polymeric layer and the conductive features are held in place, while the second polymeric layer is applied.

After providing the structures shown in FIGS. 1A and 1B, those two structures (10 and 20) are brought into intimate contact with each other such that the plurality of conductive features 24 will be transferred to the surface of the first polymeric layer 14. Next, the contacted structures are bonded together. Bonding, which can be achieved in the presence or absence of an applied external force, is performed utilizing a nominal room temperature bonding process. By “nominal room temperature” it is meant a bonding temperature from about 20° C. to about 40° C. is used. The bonding can be performed in air, under vacuum, or in an inert gas ambient.

In some embodiments of the present invention, the first polymeric layer 14 on the surface of the handle substrate 12 is treated prior to bonding to activate the surface of the first polymeric layer 14. When this treatment is performed, the structure shown in FIG. 1A is subjected to an oxygen plasma that activates the polymeric surface and promotes the adhesive of the plurality of conductive features 24 to the first polymeric layer 14. The oxygen plasma treatment is performed at a radio frequency (RF) power from about 75 to about 200 Watts using an oxygen flow from about 25 to about 100 sccm. The plasma treatment is performed for a time period from about 5 to about 10 minutes.

After bonding the two structures together, at least the carrier substrate 22 is removed by peeling off the bonded components. In some embodiments, the bonded structure can be removed from the carrier substrate 22 by utilizing a conventional lift-off procedure. The resultant structure after bonding and removal of the carrier substrate 22 is shown in FIG. 1D.

FIG. 1E shows the structure after forming a second implantable and biocompatible polymeric layer 32 on the bonded structure such that the plurality of conductive features 24 is surrounded, i.e., encased, within the two polymeric layers. The second implantable and biocompatible polymeric layer 32 may be comprised of the same or different, preferably the same, polymeric material as that of the first polymeric layer 14. In a highly preferred embodiment, polymeric layers 14 and 32 are both silicone polymers, with PDMS being most preferred. As shown, the second polymeric layer 32 includes a plurality of vias 34 which extend down through the second polymeric layer 32 and provide contact openings where a conductive material can be formed. The vias 34 expose some of the underlying conductive features 24, e.g., the metallic pads and the metallic electrodes 30. Thus, the vias 34 are formed in preselected locations within the inventive structure.

The structure shown in FIG. 1E can be formed by first applying a blanket layer of the second polymeric layer 32 to the structure shown, for example, in FIG. 1D. The blanket layer of the second polymeric layer 32 can be deposited utilizing one of the above mentioned deposition processes that was used in forming the first polymeric layer 14. The vias 34 are formed into the second polymeric layer 32 by photolithography and etching. In some embodiments of the present invention and prior to forming the photoresist on the surface of the second polymeric layer 32, the second polymeric layer 32 may be subjected to an oxygen plasma treatment which allows the resist to wet the polymeric surface preventing beading and ensuring formation of smooth and uniform resist coating on the second polymeric layer 32.

In an alternative embodiment, the structure shown in FIG. 1E is formed by providing a pre-patterned second polymeric layer 32 that contains said vias on a carrier substrate. This pre-patterned structure is formed by first applying the second polymeric layer 32 to a carrier substrate, subjecting the second polymeric layer 32 to photolithography and etching. This structure is then bonded to the structure shown in FIG. 1D utilizing the bonding conditions mentioned above. The blanket layer of second polymeric material can be subjected to oxygen plasma prior to photoresist application and a second treatment with oxygen plasma may occur after patterning the vias therein.

A conductive material 36 is then filled into the vias 34 utilizing a conventional deposition process and following deposition any conductive material outside the vias can be removed utilizing a conventional planarization process. The filled vias allow for the inventive microelectrode array shown in FIG. 2 to be interfaced with other components of the implantable medical devices including, for example, an energy source and a sensor. It is noted that the sidewalls of the vias provide openings to make contact to the electrodes.

The above steps of the present invention can be repeated numerous times to create multiple layers of metal with alternating polymeric layers to produce multi-layer three-dimensional stacks with increased number of electrodes. After all the metal and polymeric layers are formed, the devices are sectioned and removed from the carrier substrate utilizing conventional techniques well known in the art.

As stated above, the inventive microelectrode array is suitable for use as a component in an implantable medical device. Such implantable medical devices include, for example, cochlear implants, visual prostheses, neurostimulators, muscular stimulators, and deep brain stimulators. Although the inventive microelectrode array is specifically mentioned to be suitable for use in an implantable medical device, it can also find uses in electronic devices other than implantable medical devices. Other applications for the inventive microelectrode array include, but are not limited to: electrodes and electrical interconnects for medical devices that are not implanted, consumer electronics subjected to water immersion or splashing, and underwater sensing systems.

It is observed that the method of the present invention has several advantages over prior art techniques used in forming microelectrode arrays. First, the inventive method fabricates a microelectrode array with relatively thick conductive features that are flexible, stretchable and rugged. Moreover, the metallic pads, traces, and electrodes are made using a single continuous metallic sheet or foil simplifying the overall process and eliminating potential problems associated with depositing Pt or another conductive metal or a separate metal film. The inventive process is simple and low cost, and enables the fabrication of microelectrode arrays with 100's to 1000's of electrodes. In addition, the inventive method takes advantage of well-characterized manufacturing techniques (such as, for example, laser machining of stents and photolithography) and lends itself well to mass production.

While the present invention has been particularly shown and described with respect to preferred embodiments thereof, it will be understood by those skilled in the art that the foregoing changes in forms and details may be made without departing from the spirit and scope of the present application. It is therefore intended that the present invention not be limited to the exact forms and details described and illustrated herein, but fall within the scope of the appended claims.

What is claimed is:

1. A method of forming a microelectrode array for use as an element in implantable medical device comprising:
providing a bonded structure including a first structure comprising at least a first implantable and biocompatible polymeric layer and a second structure comprising a plurality of conductive features including metallic contact pads, metallic traces, and metallic electrodes, wherein each metallic trace has a zigzag pattern and substantially rounded corners; and
forming a second implantable and biocompatible polymeric layer to said bonded structure, said second polymeric layer covering said plurality of conductive features and has vias therein that extend down to said metallic contact pads and said metallic electrodes.

2. The method of claim 1 wherein said first and second polymeric layers are comprised of a same or a different polymeric material, said polymeric material selected from the group consisting of a silicone polymer, a polyurethane, a polyamide, parylene, a fluoropolymer, a polyolefin, collagen, chitin, alginate, polyvinyl pyrrolidone, polyethylene glycol, polyethylene oxide, polyvinyl alcohol, polyglycol lactic acid, polylactic acid, polycaprolactone, polyamino acid, and a hydrogel.

3. The method of claim 2 wherein both said first and second polymeric layers are comprised of a silicone polymer.

4. The method of claim 3 wherein said silicone polymer is poly(dimethylsiloxane).

5. The method of claim 1 wherein said plurality of conductive features are comprised of a conductive metal or metal alloy selected from the group consisting of Pt, Ti and NiTi.

6. The method of claim 5 wherein said conductive metal or metal alloy is Pt or NiTi.

7. The method of claim 1 wherein said providing said bonded substrate comprises a nominal room temperature bonding process and contacting of said first structure to said second structure such that an exposed surface of said first polymeric layer is in contact with an exposed surface of said plurality of conductive features.

8. The method of claim 1 wherein said plurality of conductive features is formed by laser etching a metallic sheet or foil or photolithography and etching of a metallic sheet or foil.

9. The method of claim 1 wherein said steps of bonding and forming are repeated to form a multi-layered 3D microelectrode array.

10. The method of claim 1 further comprising forming a conductive material within said vias.

11. A microelectrode array for use as an element in an implantable medical device comprising at least first and second implantable and biocompatible polymeric layers in which a plurality of patterned conductive features including metallic contact pads, metallic traces and metallic electrodes is sandwiched therebetween, wherein each metallic trace has a zigzag pattern and substantially rounded corners.

12. The microelectrode array of claim 11 wherein said first and second polymeric layers are comprised of a same or a different polymeric material, said polymeric material selected from the group consisting of a silicone polymer, a polyurethane, a polyamide, parylene, a fluoropolymer, a polyolefin, collagen, chitin, alginate, polyvinyl pyrrolidone, polyethylene glycol, polyethylene oxide, polyvinyl alcohol, polyglycol lactic acid, polylactic acid, polycaprolactone, polyamino acid, and a hydrogel.

13. The microelectrode array of claim 12 wherein both said first and second polymeric layers are comprised of a silicone polymer, a polyurethane, a polyamide, parylene, a fluoropolymer, a polyolefin, collagen, chitin, alginate, polyvinyl pyrrolidone, polyethylene glycol, polyethylene oxide, polyvinyl alcohol, polyglycol lactic acid, polylactic acid, polycaprolactone, polyamino acid, and a hydrogel.

14. The microelectrode array of claim 13 wherein said silicone polymer is poly(dimethylsiloxane).

15. The microelectrode array of claim 11 wherein said plurality of conductive features are comprised of a conductive metal or metal alloy selected from the group consisting of Pt, Ti and NiTi.

16. The microelectrode array of claim 15 wherein said conductive metal or metal alloy is Pt or NiTi.

17. The microelectrode array of claim 11 further comprising a plurality of conductively filled vias in said second polymeric layer that expose said metallic contact pads and said metallic electrodes.

18. The microelectrode array of claim 11 wherein said zigzag pattern contains from about 2 to about 200 turns and angles therein.

19. The microelectrode array of claim 11 further comprising additional implantable and biocompatible polymeric layers atop the second polymeric layer, wherein said plurality of conductive features is also present between each of said polymeric layers.

20. An implantable medical device comprising at least first and second implantable and biocompatible polymeric layers in which a plurality of patterned conductive features including metallic contact pads, metallic traces and metallic electrodes is sandwiched therebetween, wherein each said metallic trace has substantially rounded corners and a zigzag pattern and said second polymeric layer has conductively filled vias that extend down to said metallic contact pad and said metallic electrode.
